(19) United States
(12) Patent Application Publication
(10) Pub. No.: US 2003/0078541 A1
(43) Pub. Date: Apr. 24, 2003

(54) Device for injection of antiparasitic agents in pets

(76) Inventors: Udo Mattern, Stans (CH); Claudia Mattern, Stans (CH)

Correspondence Address: MERCHANT & GOULD PC, P.O. BOX 2903, MINNEAPOLIS, MN, 55402-0903, US

(21) Appl. No.: 10/148,698

(22) PCT Filed: Dec. 4, 2000
(86) PCT No.: PCT/DE00/04316

(51) Int. Cl. A61M 5/00

(52) U.S. Cl. 604/117

(57) ABSTRACT

Apparatus for injecting an active substance formulation in domestic animals, in which the apparatus comprises an injection device designed in such a way that the hypodermic needle penetrates the upper, but not the lower skin layers, and an active substance formulation with an antiparasitically active substance in a formulation suitable for injection purposes.

The invention relates to an apparatus for the application or administration of antiparasitic substances to domestic animals. The invention more particularly relates to an injection device for injectable, antiparasitically acting medicinal substances enabling the latter to be applied intradermally.

EP 897 728 A1 discloses an injection device in the form of a pin, with which an adjustable liquid quantity present in a storage container in the pin, is removed from the latter by moving a pushbutton in the longitudinal direction of the pin and can be injected into a living being through a cannula. The disclosure more particularly deals with a reliable retention of the storage container, e.g. a medicament phial, and the construction of the adjustable dosing device.

U.S. Pat. No. 5,964,731 discloses a retention support for a lancet or hypodermic needle, which makes it possible for the part appropriate for perforation to project from a sleeve and then be retracted into the sleeve again after use. In addition, the part suitable for perforation is covered in the retracted position by a cover located within the sleeve. According to this disclosure the cover, which is inverted over the needle or lancet retracted into the sleeve after use, is designed in such a way that said process is irreversible and the device cannot be used a second time.

WO 99/27984 discloses an injection device for medical use with which in particular pasty fluids can be pressed by means of an electric drive out of the storage cylinder by a plunger and can be supplied to a hypodermic needle.

Known injection devices are suitable for the injection of liquid or pasty medicinal substance formulations, the does being adjustable or can be fixed by the construction. The in each case disclosed lengths of the hypodermic needles are suitable for intramuscular or intravenous injection. As opposed to this, the present invention proposes a planned injection of the substance formulation in specific skin layers directly below the skin surface of the animal.

Antiparasitic substances are administered either orally/intramuscularly to animals, or are applied externally to the skin. Externally applied, antiparasitic substances can diffuse through the skin layers of the animal and are subsequently systemically distributed within the animal, provided that they have a relatively low molecular weight, are sufficiently lypophilic and are applied in a formulation with permeation-increasing substances, such as e.g. 2-dimethyl sulphoxide, 2-phyrrolidone, ethanol, urea, etc. as sorption providing agents.

A disadvantage of external application is that the antiparasitic substance must be formulated with a large proportion of sorption providing agent in order to render permeable for the active substance the diffusion barrier of the skin and as a result the natural skin function is destroyed. In addition, in the case of external application the formulation must be used in excess, because only a proportion of the antiparasitic substance applied actually diffuses through the skin and the remaining part is passed into the environment as a pollutant and without having any effect on the animal. The essential factors influencing the proportion of the applied, antiparasitic formulation diffusing into the skin layer are the specific state of the treated skin region, i.e. the specific characteristics of a particular body area, the age and moistness of the skin and the individually differing characteristics of the skin. It is therefore difficult to estimate for a particular animal the quantity of the antiparasitic formulation necessary for obtaining the appropriately absorbed dose of the antiparasitic substance.

In view of this prior art, the problem of the present invention is to provide an apparatus enabling an antiparasitic substance to be administered to an animal in a reliably constant or adjustable dosage.

The solution provided by the invention is to offer a combination of an injection device with an antiparasitic substance in a formulation suitable for injection. In particularly preferred manner the injection device is of the pin type and the delivered active substance formulation quantity can be dosed. It is also important for the present invention that the penetration depth of the hypodermic needle in the injection device is dimensioned in such a way that the active substance formulation is only injected directly below the top skin layers and reaches the lower skin regions, but is not injected into the subcutaneous adipose or muscle tissue. Thus, as a possible embodiment are suitable injection devices of the pin type, in which the available hypodermic needle length only projects from the pin casing by three, preferably two and in particularly preferred manner 1 mm, so that the hypodermic needle penetration depth is predetermined by the actual apparatus.

The antiparasitic substance formulation suitable for injection preferably only contains a small proportion of adjuvants, such as e.g. resorption providers, so that even a small amount of antiparasitic formulation is adequate for administering an adequate antiparasitic agent dose. Preference is given to antiparasitic formulations, in which the antiparasitic agent is suitable for injection into an animal and which is also contained in a small formulation volume. Particular preference is given to formulations, where there is an adequate antiparasitic agent dose necessary per kilogram of animal weight in a volume of 100, preferably 50 and in particularly preferred manner 10 μl.

The dosage of the antiparasitic formulation can be performed with the apparatus combination of the invention in such a way that the quantity administered per injection pulse can be adjusted to the animal weight, e.g. by a dosing device according to EP 897 728, or the constructionally fixed dosing volume of the injection device is dimensioned in such a way that it is fixed for 1, 2 or preferably 5 kg of animal weight and by multiple antiparasitic formulation dose.

The apparatus according to the invention, which comprises a combination of an injection device and an antiparasitic substance in a suitable formulation, is firstly characterized by a precise, reproducible dosage of the formulation, because there are no losses due to premature evaporation or inadequate sorption by the skin. It is also possible for the apparatus according to the invention to be safely used by non-medical practitioners, because the injection device only permits a limited hypodermic needle penetration depth, so that there is only a very limited injury risk for the animal.

It is particularly advantageous that the present invention obviates the use of large proportions of sorption providers in the active substance formulation and therefore avoids the undesired effects thereof.

The invention also makes it possible to incorporate into the formulation antiparasitic substances which, as a result of their solubility and/or inadequate lipophilicity or high molecular weight, and unsuitable for external application and sorption through the skin.

The invention also makes it possible to inject specific formulations into the skin layers of the animal which act as a depot for the antiparasitic substance.

It is clear to the expert that there can be various combinations and subcombinations of an injection device and an antiparasitic substance formulation which lead to the apparatus according to the invention.

1. Apparatus for injection an active substance formulation to domestic animals, characterized in that the apparatus comprises an injection device designed in such a way that the hypodermic needle penetrates the upper, but not the lower skin layers, and an active substance formulation with an antiparasitically active substance in a formulation suitable for injection purposes.

2. Apparatus according to claim 1, characterized in that the hypodermic needle penetration depth is max 3 mm.

3. Apparatus according to claim 1, characterized in that the hypodermic needle penetration depth is max 2 mm.

4. Apparatus according to claim 1, characterized in that the hypodermic needle penetration depth is max 1 mm.

5. Apparatus according to one of the preceding claims, characterized in that the injection device can deliver a fixed active substance formulation volume.

6. Apparatus according to one of the claims 1 to 4, characterized in that the injection device has an adjustable dosing mechanism.

7. Apparatus according to one of the preceding claims, characterized in that the injectable active substance formulation contains an antiparasitic substance from the group comprising IGR chitin synthesis inhibitors, phosphoric acid esters, carbamates, pyrethrum and pyrethroids, avarmectins, benzimidazoles, phenothiazines and praziquantel.
